(19) United States
(12) Patent Application Publication
(10) Pub. No.: US 2004/0097746 A1
(43) Pub. Date: May 20, 2004

(54) Catalyst and process for the oxidation of hydrocarbons to epoxides

(76) Inventors: Markus Dugal, Kempen (DE); Andreas Wegner, Mulheim (DE)

Correspondence Address: BAYER POLYMERS LLC, 100 BAYER ROAD, PITTSBURGH, PA, 15205, US

(21) Appl. No.: 10/698,683

(22) Filed: Oct. 31, 2003

(30) Foreign Application Priority Data
Nov. 5, 2002 (DE) 10251325.2

(51) Int. Cl. B01J 23/34; C07D 301/06

(52) U.S. Cl. 549/533; 502/324

(57) ABSTRACT

The present invention concerns a catalyst containing manganese in elemental or in combined form and containing an element selected from lithium, sodium, potassium, rubidium, cesium, beryllium, magnesium, calcium, strontium, barium and zinc in elemental or combined form together with a process for producing this catalyst and a process for oxidizing hydrocarbons to epoxides in the presence of the inventive catalyst.

### FIELD OF THE INVENTION

The present invention concerns a catalyst containing manganese in elemental or in combined form and containing an element selected from lithium, sodium, potassium, rubidium, cesium, beryllium, magnesium, calcium, strontium, barium and zinc in elemental or combined form together with a process for producing this catalyst and a process for oxidizing hydrocarbons to epoxides in the presence of this catalyst.

### BACKGROUND OF THE INVENTION

Epoxides are an important starting material for the polyurethane industry. There are a number of processes developed for producing them, some of which have been industrialized. For the industrial production of ethylene oxide, the direct oxidation of ethene with air or with gases containing molecular oxygen is used in the presence of a silver-containing catalyst. This is described in EP-A 0 933 130.

To produce epoxides having more than two carbon atoms, hydrogen peroxide and/or hypochlorite are generally used on an industrial scale as oxidizing agents in the liquid phase. EP-A 0 930 308 for example describes the use of ion-exchanged titanium silicalites as catalyst with these two oxidizing agents.

Another class of oxidation catalysts, which allows propene to oxidize to propene oxide in the gas phase, is disclosed in U.S. Pat. No. 5,623,090. Here gold on anatase is used as the catalyst and the oxidizing agent is oxygen, which is used in the presence of hydrogen. The system is characterized by an unusually high selectivity (S>95%) with regard to propene oxidation. The disadvantages are the low conversion and the deactivation of the catalyst.

DE-A 100 24 096 discloses that mixtures containing manganese and at least one other element selected from the group consisting of Cu, Ru, Rh, Pd, Os, Ir, Pt, Au, In, Ti and Ce can catalyze the direct oxidation of propene to propene oxide. DE-A 101 39 531 and DE-A 102 08 254 likewise disclose catalysts for the oxidation of propene to propene oxide.

### SUMMARY OF THE INVENTION

The present invention provides catalysts for the oxidation of hydrocarbons to epoxides and provides a process for the oxidation of hydrocarbons in the presence of these catalysts.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.” The present invention provides a catalyst made of

a) a support

b) manganese in elemental or in combined form and

c) one or more different elements selected from lithium, sodium, potassium, rubidium, caesium, beryllium, magnesium, calcium, strontium, barium and zinc in elemental or combined form,

wherein the support has a BET surface area of less than 200 m²/g.

The present invention also provides a process for producing the inventive catalyst by the steps of

a) providing a support having a BET surface area of less than 200 m²/g,

b) impregnating the support with one or more different solutions, which taken together contain manganese and one or more compounds of one or more elements selected from lithium, sodium, potassium, rubidium, caesium, beryllium, magnesium, calcium, strontium, barium and zinc,

c) drying the impregnated support,

d) calcining the dried support.

The present invention also provides a process for producing an epoxide from a hydrocarbon by the reaction of the hydrocarbon with an oxygen-containing gaseous oxidizing agent in the presence of the inventive catalyst containing manganese in elemental or in combined form and one or more different elements selected from lithium, sodium, potassium, rubidium, caesium, beryllium, magnesium, calcium, strontium, barium and zinc in elemental or combined form.

A particularly preferred process of the present invention is one in which the oxidizing agent is selected from oxygen and nitrogen oxides.

A particularly preferred process of the present invention is one in which the inventive catalyst is also used.

The catalyst of the present invention and the inventive process have many advantages. The catalyst has a high activity and it has a high selectivity in the oxidation of hydrocarbons to epoxides, particularly in the oxidation of propene to propene oxide.

The oxidation of a hydrocarbon by the process according to the present invention or in the presence of the catalyst according to the present invention ends at the epoxide stage and does not lead completely to the corresponding acid or to the aldehyde or ketone.

In one embodiment, the catalyst of the present invention has one or more different elements selected from sodium, potassium and cesium.

In another embodiment, the catalyst according to the present invention has manganese in elemental or in combined form and sodium in combined form.

In another embodiment, the catalyst according to the present invention has manganese in elemental or in combined form and potassium in combined form.

In the present disclosure, the term hydrocarbon means unsaturated or saturated hydrocarbons such as olefins or alkanes. These can also contain heteroatoms such as N, O, P, S or halogens.

These hydrocarbons can be acyclic, monocyclic, bicyclic or polycyclic. These hydrocarbons can be monoolefinic, diolefinic or polyolefinic.

These hydrocarbons can contain two or more double bonds. In this case the double bonds can be conjugated and non-conjugated.

Preferred hydrocarbons are those from which such oxidation products are formed whose partial pressure at the reaction temperature is low enough to remove the product continuously from the catalyst.

Unsaturated or saturated hydrocarbons having 2 to 20, preferably 3 to 10 carbon atoms are preferred.

Hydrocarbons selected from the group consisting of propene, propane, isobutane, isobutylene, 1-butene, 2-butene, cis-2-butene, trans-2-butene, 1,3-butadiene, pentene, pentane, 1-hexene, 1-hexane, hexadiene, cyclohexene and benzene are particularly preferred.

Hydrocarbons selected from propene and butene are particularly preferred. Of these propene is most particularly preferred.

Any gaseous, oxygen-containing oxidizing agents are suitable according to the invention. In one embodiment of the present invention, the gaseous, oxygen-containing oxidizing agent is selected from oxygen and nitrogen oxides. In another embodiment of the present invention, the oxidizing agent is oxygen.

A mixture with other gases can also be used as an oxidizing agent. Thus a gas mixture containing oxygen and nitrogen can be used, for example. Or air can be used.

The manganese and the element selected from lithium, sodium, potassium, rubidium, cesium, beryllium, magnesium, calcium, strontium, barium and zinc can be present in the inventive catalyst in elemental or in combined form. In a preferred embodiment of the present invention both may be present in combined form.

The ratio of manganese to alkali metal or alkaline-earth metal in the catalyst according to the invention can be varied within broad ranges. Preferred ratios are manganese to alkali metal or manganese to alkaline-earth metal of 1000 to 1 to 1 to 10, more preferably 100 to 1 to 1 to 5. These ratios are mass ratios of the cited elements.

Catalysts according to the invention can also contain manganese and several different alkali metals and/or alkaline-earth metals and zinc.

It can be advantageous if the catalyst according to the invention additionally contains promoters or moderators, for example other alkaline-earth metals and/or alkali metals and/or Zn as hydroxides, carbonates, nitrates, chlorides, carboxylates, alcoholates, acetates or in the form of other salts and/or silver (in elemental or in combined form). Suitable promoters are described in EP-A 0 933 130 on page 4, line 39 ff.

The manganese in elemental or in combined form and the element selected from lithium, sodium, potassium, rubidium, cesium, beryllium, magnesium, calcium, strontium, barium and zinc in elemental or in combined form and the optionally present promoters and the optionally present moderators can be present in the catalyst according to the invention in broad quantity limits.

Other embodiments of the present invention are provided where the quantity of each of the cited constituents is mutually independently within the limits 0.01 to 99.99 wt. %, preferably 0.1 to 99.9 wt. %. This refers to the quantity of the compound of manganese or of the cited element if it is not present in elemental form. The cited quantities are in wt. % relative to the sum of the quantity of manganese in elemental or in combined form and of the element selected from lithium, sodium, potassium, rubidium, cesium, beryllium, magnesium, calcium, strontium, barium and zinc in elemental or combined form and of the promoters and moderators.

The preferred range for the quantity of promoters in the catalyst according to the invention is 0.001 to 35 wt. %, relative to the sum of the quantity of manganese in elemental or in combined form and of the element selected from lithium, sodium, potassium, rubidium, cesium, beryllium, magnesium, calcium, strontium, barium and zinc in elemental or combined form and of the promoters and moderators.

The catalyst according to the invention may or may not include a support. Catalysts without supports may be produced by various processes. They can for example be produced by the thermal degradation of metal salts, or by precipitation processes and by a sol-gel process.

In an embodiment of the present invention, the catalyst includes a support. In that embodiment, the support according to the invention is selected from Al₂O₃, SiO₂, CeO₂, ZrO₂, SiC and TiO₂.

In a preferred embodiment of the present invention, the support is Al₂O₃.

In an embodiment of the present invention, the support has a BET surface area of less than 200 m²/g, preferably less than 100 m²/g, more preferably less than 10 m²/g, and most preferably less than 1 m²/g.

The BET surface area is measured by the method according to Brunauer, Emmet and Teller.

The BET surface area of the support is measured before the support is coated with manganese, alkali metals or alkaline-earth metals or other substances.

The BET surface area is determined in the conventional way according to Brunauer, Emmet and Teller, Journal of the American Chemical Society, 1938, volume 60, page 309 (and according to DIN 66 131).

In another embodiment of the present invention, the support is porous.

The porosity of the support is preferably 20 to 60% (part by volume of the support), more preferably 30 to 50%. The porosity can be determined in the conventional way, for example using mercury porosimetry.

The particle size of the support according to the invention can vary within broad ranges. It is chosen according to the process conditions for the oxidation of the hydrocarbons. It is preferably in the range from 1/10 to 1/20 of the reactor diameter.

The particle size of the particles containing manganese on the surface of the support can be determined by electron microscopy and X-ray diffractometry.

The sum of the masses of manganese or manganese compounds and of the elements or element compounds (in other words the element selected from lithium, sodium, potassium, rubidium, cesium, beryllium, magnesium, calcium, strontium, barium and zinc) on the support should preferably be in the range from 0.001 to 50 wt. %, more preferably 0.001 to 20 wt. %, and most preferably 0.01 to 10 wt. % (relative to the sum of the masses of the support and this mass).

Processes with which the catalyst according to the invention can be produced are described below, wherein the manganese in elemental or in combined form and one or more elements selected from lithium, sodium, potassium, rubidium, cesium, beryllium, magnesium, calcium, strontium, barium and zinc in elemental or combined form are applied to the support according to the invention. The term “element according to the invention” hereafter refers to one or more different elements selected from lithium, sodium, potassium, rubidium, cesium, beryllium, magnesium, calcium, strontium, barium and zinc in elemental or combined form. For the sake of simplicity only “manganese” is referred to below. In each case this means manganese in elemental or combined form.

In one embodiment of the present invention the manganese is present in the catalyst according to the invention as oxide.

In a preferred embodiment of the present invention the element(s) according to the invention is/are present in the catalyst according to the invention as oxide.

Production of particles of manganese and the element according to the invention on the support is not restricted to any one method. Processes that can be used include for example:

the deposition-precipitation process, such as disclosed for example in EP-B-0 709 360 on page 3, lines 38 ff.,

impregnation in solution,

the incipient wetness process,

the colloid process,

sputtering,

CVD (chemical vapor deposition) and

PVD (physical vapor deposition).

The incipient wetness process refers to the addition of a solution containing soluble compounds of manganese and of the element according to the invention to the support, the volume of the solution being less than or equal to the pore volume of the support. The support thus remains macroscopically dry.

All solvents in which the compounds of manganese and of the element according to the invention are soluble can be used as solvent for the incipient wetness process. Suitable solvents are for example water, alcohols, (crown) ethers, esters, ketones, halogenated hydrocarbons, etc.

The support is preferably impregnated with a solution containing compounds of manganese and of the element according to the invention and then dried and calcined. This solution can additionally contain components known to the person skilled in the art that can increase the solubility of the compounds of manganese and of the element according to the invention in the solvent and/or change the redox potentials of the manganese and/or of the element according to the invention and/or change the pH. Ammonia, amines, diamines, hydroxyamines and acids such as HCl, HNO₃, H₂SO₄, H₃PO₄ can be cited as non-limiting examples of such components.

Impregnation of the support with a solution containing the compounds of manganese and of the element according to the invention can be performed after the incipient wetness process, for example.

The incipient wetness process may include the following steps:

coating the support once with compounds of manganese and/or of the element according to the invention and/or coating the support repeatedly with other compounds of manganese and/or of the element according to the invention,

coating once with part of the compounds of manganese and of the element according to the invention or with the entire amount of the compounds of manganese and of the element according to the invention in one step,

coating repeatedly with several compounds of manganese and several compounds of the element according to the invention successively in one or more steps,

coating repeatedly with several compounds of manganese and several compounds of the element according to the invention alternately in one or more steps.

Drying of the impregnated support obtained after impregnation is performed preferably at a temperature of approximately 40° C. to approximately 200° C. under normal pressure or alternatively under reduced pressure. Under normal pressure it is possible to operate under an air atmosphere or alternatively under an inert gas atmosphere (e.g. Ar, N₂, He or other inert gases). The drying time is preferably in the range from 2 to 24 hours, more preferably-from 4 to 8 hours.

Calcining of the dried support obtained after drying can be performed first under an inert gas atmosphere and then under an oxygen-containing gas atmosphere. It can also be performed exclusively under an oxygen-containing gas atmosphere. The content of oxygen in the cited gas atmosphere is preferably in the range from 0 to 30 vol. %, more preferably 5 to 21 vol. % (relative to the volume of the gas atmosphere).

Calcining of the dried support obtained after drying can be performed in air at temperatures of 20 to 1000° C. The temperature range of 300 to 700° C. is preferred for calcining in air.

The temperature for calcining is chosen differently according to the element that is used. It is in the range from 200 to 1000° C., preferably 300 to 900° C., more preferably 350 to 550° C., most preferably around 400° C.

Reduction of the catalyst precursor (support) takes place in particular at elevated temperature under a nitrogen atmosphere containing hydrogen. The content of hydrogen can preferably be between 0 and 100 vol. %, more preferably 0 to 25, most preferably 1 to 10 vol. % (relative to the volume of the entire nitrogen atmosphere). The reduction temperatures are adjusted to the individual element and are preferably between 100 and 600° C.

Various manganese compounds can be used as starting compounds for the manganese contained in the catalyst according to the invention, depending on the method of synthesis. For example, manganese halides, manganese acetates, manganese nitrates, manganese carboxylates, manganese alcoholates, manganese sulfates, manganese phosphates, manganese hydroxides, manganese acetyl acetonates, manganese oxides, manganese carbonates or manganese amine complexes can be used. The manganese can be present in a wide range of oxidation stages.

Various alkali and/or alkaline-earth metal compounds can be used as starting compounds for the alkali and/or alkaline-earth metals in the catalyst according to the invention. For example, nitrates, halides, carboxylates, carbonates, hydrogen carbonates, hydroxides, oxides, acetates, acetyl acetonates, alcoholates, phosphates or sulfates can be used.

The process according to the present invention for the production of an epoxide from a hydrocarbon is described below.

The process is preferably performed under the following conditions:

It is preferably performed in the gas phase.

The molar quantity of the hydrocarbon used relative to the total number of moles of hydrocarbon, oxygen and of diluent gas that is optionally present and the relative molar ratio of the components can be varied in broad ranges and is governed by the explosive limits of the hydrocarbon-oxygen mixture. The process is preferably performed above or below the explosive limit outside the explosive range.

An excess of hydrocarbon, relative to the oxygen used (on a molar basis), is preferably used. The hydrocarbon content in the reaction gas is preferably ≦2 mol % or ≧78 mol % (relative to the sum of all moles in the reaction gas). Hydrocarbon contents in the range from 0.5 to 2 mol % are preferably chosen for operations below the lower explosive limit and from 78 to 99 mol % for operations above the upper explosive limit. The ranges from 1 to 2 mol % and 78 to 90 mol % respectively are particularly preferred.

The molar oxygen content, relative to the total number of moles of hydrocarbon, oxygen and diluent gas, can be varied in broad ranges. The molar quantity of oxygen used is preferably less than that of hydrocarbon. Oxygen is preferably used in the range from 1 to 21 mol %, more preferably 5 to 21 mol % (relative to the total moles in the gas stream).

In addition to hydrocarbon and oxygen, a diluent gas such as nitrogen, helium, argon, methane, carbon dioxide, carbon monoxide or similar predominantly inert gases can optionally be used. Mixtures of the inert components described can also be used. The addition of inert components is favorable for the transport of heat released by this exothermic oxidation reaction and from a safety perspective. In this case the composition of reactant gas mixtures described above is also possible in the explosive range of the undiluted mixture of hydrocarbon and oxygen.

The contact time between hydrocarbon and catalyst is preferably in the range from 0.1 to 100 seconds, more preferably in the range from 5 to 60 seconds.

The process is preferably performed at temperatures in the range from 120 to 300° C., more preferably 160 to 260° C.

### EXAMPLES

The examples serve to illustrate the invention. The scope of the invention is not limited to the examples.

### Examples 1 to 22

In examples 1 to 16 so-called stock solutions were used. These stock solutions were produced as follows. For solution 1, 40.09 g manganese(II) nitrate were dissolved in 64.2 g distilled water. For solution 2, 32.4 g sodium nitrate were dissolved in 75 g distilled water.

Mixtures of these solutions with a total volume of 2.39 ml were completely imbibed in 5 g Al₂O₃. The solid thus obtained was dried for 8 hours at 100° C. in a vacuum drying cabinet under a vacuum of approx. 20 mbar. A so-called precursor of the catalyst was obtained in this way.

Finally, the precursor produced in this way was aftertreated in various ways for 8 hours. This aftertreatment was performed either by calcining in the presence of air (method 1 in Table 1) or by reduction in a gas mixture comprising 10 vol. % H₂ and 90 vol. % N₂, relative in each case to the total volume of gas, at a flow rate of 60 l/h (method 2 in Table 1). The aftertreatment temperatures are set out in Table 1. The catalyst was obtained by the aftertreatment.

Following the aftertreatment, 1 g of the catalyst thus obtained was analyzed in a continuous fixed-bed reactor for a residence time of approx. 20 seconds with a gas mixture having a composition of 79 vol. % propene and 21 vol. % oxygen. The reaction temperature stated in Table 1 is the reactor temperature in the fixed bed. The results of these analyses can likewise be found in Table 1.

TABLE 1
Production of the catalysts and suitability
of the catalysts for the oxidation of
propene to propene oxide (PO).
(Selectivity is defined as the number of
carbon atoms in propene oxide divided by
the number of carbon atoms in all
carbon-containing reaction products)

| | | | After- | | | |
|---|---|---|---|---|---|---|
| | | | treatment | Reaction | PO | |
| | | | (method/ | tem- | content in | Selec- |
| | Solution | Solution | temperature | perature | waste gas | tivity |
| No. | 1 [ml] | 2 [ml] | in ° C.) | in ° C. | in ppm | in % |
| 1 | 1.196 | 1.196 | 1/400 | 250 | 1998 | 16 |
| 2 | 0.797 | 1.594 | 1/400 | 250 | 1365 | 13 |
| 3 | 0.598 | 1.794 | 1/400 | 250 | 1007 | 11 |
| 4 | 1.594 | 0.797 | 1/400 | 250 | 1404 | 13 |
| 5 | 0.957 | 1.435 | 1/400 | 240 | 1202 | 13 |
| 6 | 1.794 | 0.598 | 1/400 | 250 | 1224 | 12 |
| 7 | 1.435 | 0.957 | 1/400 | 250 | 961 | 11 |
| 8 | 1.196 | 1.196 | 1/500 | 240 | 537 | 7 |
| 9 | 0.797 | 1.594 | 1/500 | 250 | 130 | 2 |
| 10 | 0.598 | 1.794 | 1/500 | 250 | 44 | 1 |
| 11 | 1.594 | 0.797 | 1/500 | 250 | 245 | 16 |
| 12 | 1.794 | 0.598 | 1/500 | 240 | 508 | 4 |
| 13 | 1.435 | 0.957 | 1/500 | 250 | 138 | 12 |
| 14 | 1.196 | 1.196 | 2/400 | 250 | 110 | 1 |
| 15 | 1.794 | 0.598 | 2/500 | 240 | 193 | 1 |
| 16 | 1.594 | 0.797 | 2/400 | 250 | 148 | 1 |
| 17 | 0.797 | 1.594 | 1/300 | 240 | 1260 | 22 |
| 18 | 0.797 | 1.594 | 1/300 | 185 | 192 | 32 |
| 19 | 1.794 | 0.598 | 1/300 | 240 | 1914 | 5 |
| 20 | 0.598 | 1.794 | 1/350 | 180 | 177 | 64 |
| 21 | 1.594 | 0.797 | 1/350 | 250 | 1935 | 27 |
| 22 | 1.794 | 0.598 | 1/350 | 250 | 1949 | 26 |

### Examples 23 to 28

In examples 23 to 28 so-called stock solutions were used. These stock solutions were produced as follows. For solution 3, 39.15 g manganese acetate were dissolved in 63.5 g distilled water. For solution 4, 51.56 g sodium acetate were dissolved in 54.4 g distilled water.

Mixtures of these solutions with a total volume of 2.39 ml were completely imbibed in 5 g Al₂O₃. The solid thus obtained was dried for 8 hours at 100° C. in a vacuum drying cabinet under a vacuum of approx. 20 mbar. A so-called precursor of the catalyst was obtained in this way.

Finally, the precursor produced in this way was aftertreated for 8 hours by calcining in the presence of air. The aftertreatment temperatures are set out in Table 2. The catalyst was obtained by the aftertreatment.

Following the aftertreatment, 1 g of the catalyst thus obtained was analyzed in a continuous fixed-bed reactor for a residence time of-approx. 20 seconds with a gas mixture having a composition of 79 vol. % propene and 21 vol. % oxygen. The reaction temperature stated in Table 2 is the reactor temperature in the fixed bed. The results of these analyses can likewise be found in Table 2.

TABLE 2
Production of catalysts and suitability
of the catalysts for the
oxidation of propene to propene oxide (PO).

| | | | | | PO | |
|---|---|---|---|---|---|---|
| | | | After- | | content | |
| | | | treatment | Reaction | in waste | Selec- |
| | Solution | Solution | temperature | temperature | gas in | tivity |
| No. | 3 [ml] | 4 [ml] | in ° C. | in ° C. | ppm | in % |
| 23 | 0.797 | 1.594 | 300 | 235 | 2193 | 5 |
| 24 | 1.594 | 0.797 | 300 | 250 | 1646 | 22 |
| 25 | 0.957 | 1.435 | 300 | 250 | 1281 | 23 |
| 26 | 1.794 | 0.598 | 500 | 240 | 275 | 3 |
| 27 | 0.957 | 1.435 | 400 | 240 | 134 | 29 |
| 28 | 1.794 | 0.598 | 400 | 240 | 553 | 6 |

### Examples 29 to 37

In examples 29 to 37 so-called stock solutions were used. These stock solutions were produced as follows. For solution 5, 40.09 g manganese nitrate were dissolved in 64.2 g distilled water. For solution 6, 22.7 g potassium nitrate were dissolved in 75 g distilled water.

Mixtures of these solutions with a total volume of 2.39 ml were completely imbibed in 5 g Al₂O₃. The solid thus obtained was dried for 8 hours at 100° C. in a vacuum drying cabinet under a vacuum of approx. 20 mbar. A so-called precursor of the catalyst was obtained in this way.

Finally, the precursor produced in this way was aftertreated in various ways for 8 hours. This aftertreatment was performed either by calcining in the presence of air (method 1 in Table 3) or by reduction in a gas mixture comprising 10 vol. % H₂ and 90 vol. % N₂, relative in each case to the total volume of gas, at a flow rate of 60 l/h (method 2 in Table 3). The aftertreatment temperatures are set out in Table 3. The catalyst was obtained by the aftertreatment.

Following the aftertreatment, 1 g of the catalyst thus obtained was analysed in a continuous fixed-bed reactor for a residence time of approx. 20 seconds with a gas mixture having a composition of 79 vol. % propene and 21 vol. % oxygen. The reaction temperature stated in Table 3 is the reactor temperature in the fixed bed. The results of these analyses can likewise be found in Table 3.

TABLE 3
Production of the catalysts and
suitability of the catalysts for the
oxidation of propene to propene oxide (PO).

| | | | After- | | PO | |
|---|---|---|---|---|---|---|
| | | | treatment | | content | |
| | | | (method/ | Reaction | in waste | Selec- |
| | Solution | Solution | temperature | temperature | gas in | tivity |
| No. | 5 [ml] | 6 [ml] | in ° C.) | in ° C. | ppm | in % |
| 29 | 1.196 | 1.196 | 1/500 | 250 | 953 | 20 |
| 30 | 1.196 | 1.196 | 1/300 | 250 | 2304 | 18 |
| 31 | 0.957 | 1.435 | 1/300 | 250 | 1864 | 22 |
| 32 | 0.957 | 1.435 | 1/300 | 180 | 178 | 35 |
| 33 | 1.196 | 1.196 | 1/400 | 250 | 3539 | 21 |
| 34 | 0.797 | 1.594 | 1/400 | 250 | 3777 | 20 |
| 35 | 0.598 | 1.794 | 1/400 | 250 | 2737 | 24 |
| 36 | 1.594 | 0.797 | 1/400 | 250 | 3674 | 21 |
| 37 | 0.598 | 1.794 | 2/500 | 240 | 1768 | 4 |

### Examples 38 to 44

In examples 38 to 44 so-called stock solutions were used. These stock solutions were produced as follows. For solution 7, 40.09 g manganese nitrate were dissolved in 64.2 g distilled water. For solution 8, 12.87 g cesium nitrate were dissolved in 75 g distilled water.

Mixtures of these solutions with a total volume of 2.39 ml were completely imbibed in 5 g Al₂O₃. The solid thus obtained was dried for 8 hours at 100° C. in a vacuum drying cabinet under a vacuum of approx. 20 mbar. A so-called precursor of the catalyst was obtained in this way.

Finally, the precursor produced in this way was aftertreated in various ways for 8 hours. This aftertreatment was performed either by calcining in the presence of air (method 1 in Table 4) or by reduction in a gas mixture comprising 10 vol. % H₂ and 90 vol:% N₂, relative in each case to the total volume of gas, at a flow rate of 60 l/h (method 2 in Table 4). The aftertreatment temperatures are set out in Table 4. The catalyst was obtained by the aftertreatment.

Following the aftertreatment, 1 g of the catalyst thus obtained was analyzed in a continuous fixed-bed reactor for a residence time of approx. 20 seconds with a gas mixture having a composition of 79 vol. % propene and 21 vol. % oxygen. The reaction temperature stated in Table 4 is the reactor temperature in the fixed bed. The results of these analyses can likewise be found in Table 4.

TABLE 4
Production of the catalysts and
suitability of the catalysts for the
oxidation of propene to propene oxide (PO).

| | | | After- | | PO | |
|---|---|---|---|---|---|---|
| | | | treatment | | content | |
| | | | (method/ | Reaction | in waste | Selec- |
| | Solution | Solution | temperature | temperature | gas in | tivity |
| No. | 7 [ml] | 8 [ml] | in ° C.) | in ° C. | ppm | in % |
| 38 | 0.797 | 1.594 | 1/500 | 240 | 2032 | 12 |
| 39 | 0.598 | 1.794 | 1/300 | 250 | 2186 | 13 |
| 40 | 1.196 | 1.196 | 1/400 | 250 | 2537 | 10 |
| 41 | 0.797 | 1.594 | 1/400 | 250 | 2497 | 10 |
| 42 | 0.598 | 1.794 | 1/400 | 250 | 2780 | 13 |
| 43 | 0.598 | 1.794 | 1/400 | 180 | 369 | 20 |
| 44 | 1.594 | 0.797 | 2/500 | 240 | 20 | <1 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A catalyst comprising a support;
manganese in elemental or combined form; and
one or more elements selected from the group consisting of lithium, sodium, potassium, rubidium, caesium, beryllium, magnesium, calcium, strontium, barium and zinc in elemental or combined form,
wherein the support has a BET surface area of less than 200 m²/g.

2. A process for producing the catalyst according to claim 1 comprising the steps of:
providing a support having a BET surface area of less than 200 m²/g;
impregnating the support with one or more solutions, which taken together contain manganese and one or more compounds of one or more elements selected from the group consisting of lithium, sodium, potassium, rubidium, cesium, beryllium, magnesium, calcium, strontium, barium and zinc;
drying the impregnated support; and
calcining the dried support.

3. The catalyst made by the process according to claim 2.

4. In a process for producing an epoxide from a hydrocarbon comprising reaction of the hydrocarbon with an oxygen-containing gaseous oxidizing agent in the presence of a catalyst, the improvement comprising the catalyst containing manganese in elemental or in combined form and containing one or more elements selected from the group consisting of lithium, sodium, potassium, rubidium, cesium, beryllium, magnesium, calcium, strontium, barium and zinc in elemental or combined form.

5. The process according to claim 4, wherein the oxidizing agent is selected from the group consisting of oxygen and nitrogen oxides.
